(19) United States
(12) Patent Application Publication
(10) Pub. No.: US 2006/0074303 A1
(43) Pub. Date: Apr. 6, 2006

(54) Apparatus and method for conformal radiation brachytherapy for prostate gland and other tumors

(75) Inventors: Victor I. Chornenky, Minnetonka, MN (US); Ali Jaafar, Eden Prairie, MN (US)

Correspondence Address: OFFICES OF CRAIG GREGERSEN, P.O. BOX 386353, 10032 QUEBEC AVENUE SOUTH, BLOOMINGTON, MN, 55438, US

(73) Assignee: Minnesota Medical Physics LLC

(21) Appl. No.: 11/236,065

(22) Filed: Sep. 27, 2005

Related U.S. Application Data
(60) Provisional application No. 60/613,210, filed on Sep. 28, 2004.

(51) Int. Cl. A61B 5/05; A61N 5/00

(52) U.S. Cl. 600/427; 600/1

(57) ABSTRACT

A system and method providing conformal x-ray brachytherapy for treatment of tumors by irradiation of a target volume of tissue in a patient is disclosed wherein an x-ray probe including an x-ray emitter, an imaging probe configured to image the target volume, a translation stage mounting the x-ray probe for translational motion, a rotation stage mounting the x-ray probe for rotational motion, a support base mounting the x-ray and imaging probes in known relation to each other, and a computer operatively connected to the x-ray and imaging probes and the rotation and translation stages are provided to image and control the operation of the x-ray probe to irradiate the target volume according to predetermined treatment protocols.

### BACKGROUND OF THE INVENTION

This application claims priority from U.S. Provisional Patent Application Ser. No. 60/613,210 entitled Apparatus And Method For Conformal Radiation Brachytherapy, filed Sep. 28, 2004 and from U.S. patent application Ser. No. 10/392,167 (Published Application No. 20030179854) entitled “X-Ray Apparatus With Field Emission Current Stabilization And Method Of Providing X-Ray Radiation Therapy” and filed on Mar. 19, 2003 and U.S. patent application Ser. No. 10/938,971 (Published Application No. 20050038488), also entitled “X-Ray Apparatus With Field Emission Current Stabilization And Method Of Providing X-Ray Radiation Therapy” and filed on Sep. 10, 2004.

Radiation therapy has been and will for the foreseeable future continue to be an available and oft-used treatment modality (either alone or in some combination with surgery, chemotherapy, and/or hormone therapy) for the occurrence of cancerous tumors. Examples of the types of tumors treated with radiation therapy include cancers of the prostate, the breast, the lung, and the brain, head and neck, amongst others. Typically, the radiation therapy is provided to a localized tissue area surrounding the tumor. Depending upon the type of tumor and its location, the tumor may be excised prior to radiation therapy or it may be left in place and treated with radiation also.

Broadly speaking, treatment of a body with radiation because of such tumors can occur with the use of either internal (also known as brachytherapy) or external radiation sources. Both internal and external radiation sources have their own respective advantages and disadvantages well known to practitioners. Generally in external radiation therapy, a plurality of angles of exposure are used to irradiate the tumor and/or the surrounding marginal tissue so as to provide overlapping coverage of the tumor. The effect of the overlapping coverage is to ensure that the largest radiation dose is received at the desired treatment location while minimizing the radiation damage to the surrounding tissue. For example, typical slow-growth prostate gland tumors are typically not excised prior to radiation therapy. When treating such tumors with radiation, care should be taken to avoid or minimize radiation damage to the urethra, the rectum, and the peripheral nerve bundle of the prostate gland. Damage to the latter could lead to impotence. Yet, effective treatment requires that sufficient radiation be delivered to the prostate gland to destroy the cancerous cells. As another example, breast cancers are typically excised and the margin tissue surrounding the excised tumor is treated with radiation to hopefully kill any remaining cancer cells. Were this tissue to be treated externally from a single angle, radiation burns along the beam path would almost surely result in unwanted and undesirable damage to healthy tissue.

Thus, a common element in the successful use of either an internal or external radiation source for therapy that also minimizes radiation damage is knowledge of the geometry of the desired treatment volume. Knowing the geometry of the desired treatment volume, with or without tissue excision, enables the therapist to target that treatment volume from multiple angles and to reduce thereby the exposure of surrounding tissue to radiation.

More specifically, in the last 10-15 years, a new technology in radiation therapy has improved targeting accuracy, thereby allowing higher, more effective doses to be delivered to a tumor bed while minimizing side effects and complications. This new modality of therapy uses multiple specially shaped or “modulated” beams applied from several different directions to the target volume—that volume of tissue including the tumor and surrounding tissue to be target for receipt of therapeutic x-ray radiation. The main objective of the therapy is to concentrate radiation on tumors and minimize radiation dosages applied to the adjacent healthy tissue, especially to the critical parts of the body that are more sensitive to radiation. This technology is called Intensity-Modulated Radiation Therapy (IMRT), an advanced form of external beam irradiation that is commonly referred to as three-dimensional conformal radiation therapy (3DCRT).

Several advances in medical technology made the 3DCRT possible. The most important one was the development of sophisticated 3D imaging techniques, among them computer-assisted tomography (CAT), magnetic resonance imaging (MRI), ultrasound (US), and positron emission tomography (PET).

Each of the aforementioned imaging technologies utilize different tissue properties to distinguish adjacent tissues from each other. For example, CAT scans, MRI and US use physical properties of tissues to distinguish one tissue from another while PET scans utilize metabolic differences between malignant and healthy tissues. More specifically, CAT scans utilize differences in the various tissue electron densities to distinguish one tissue type from another. MRI uses differences in the hydrogen densities of various tissues to distinguish one from the other. Ultrasound imaging, on the other hand, uses differences in the acoustic properties of tumors and surrounding tissues, which results in reflections of ultrasound waves at the boundary of two tissues having different sound transmission speeds.

Development of the CAT scans enabled three-dimensional reconstructions of a patient's anatomy with high spatial resolution. This imaging modality provides substantially better visualization of the cancer and surrounding normal tissue in three dimensions. With this comprehensive ability to identify the target volume and the surrounding normal tissues in three-dimensional space, physicians can customize the shapes of radiation beams for each patient and more precisely aim a beam into the target volume from multiple directions while substantially reducing the exposure of surrounding normal tissues to the radiation beams.

Another important modality of 3D imaging that has been significantly improved over the last decade is MRI. MRI allows better differentiation between malignant and healthy tissues and is known for providing sharp differentiations between tumors and surrounding soft tissues, for example in the brain or prostate gland. As its resolution continues to improve, MRI becomes increasingly involved in cancer diagnosis and therapy.

All these imaging modality give somewhat different 3D images of the gross tumors and disseminated micro tumors around them. They compliment each other; combined together they allow a diagnostician to compile a better diagnostic image of the tumor bed and thereby enable the physician to delineate the target volume and adjacent critical structures more precisely.

Another imaging advance is the ability to rotate an image of a patient's anatomy in 3D virtual space and, especially newly developed software called Room's-Eye-View (REV). This functionality gives radiation oncologists a tool for customizing radiation beam cross sections and directions for irradiation of the tumor that provide high conformity with an identified 3D target volume. This software tool provides an interactive three-dimensional isodose surface display, which is a valuable tool for evaluation of proposed 3D radiation therapy dose distributions in terms of ensuring adequate coverage of the target volume while sparing critical structures. The REV display enables radiation oncologists to view a target volume or a normal tissue volume with superimposed isodose surfaces or “dose clouds” from any arbitrary viewing angle. Using different multi-leaf collimators to shape the radiation beams generated by therapeutic machines oncologists have succeeded in increasing doses for malignant tumors and sparing critical structures around them thus improving the local control of the disease and decreasing toxicity not only for critical structures but for the adjacent tissues in general.

Another approach for conformal radiation therapy has been developed wherein brachytherapy is provided by implantation of radioactive seeds that covering the target volume with the desired radiation dose. This therapy modality It uses real time computation of the 3D distribution of the radiation dose received by the target volume and surrounding tissue as the oncologist places the seeds.

To achieve high quality radiation therapy, it is necessary to accurately relate the positions of target volumes and critical structures in the patient to the positions and orientation of beams used for imaging and treatment. This requires the use of multiple coordinate systems, one within the patient and those related to the imaging and treatment machines. The positions of target volumes and critical structures are related to anatomic reference points or alignment marks in the coordinate system of the patient. The position and orientation of the imaging and treatment machines are defined in the coordinate systems related to these machines. Because the reference points of the patient's anatomy and special radio opaque marks made on the patient skin can be defined in both patient and machine coordinate system, they can serve as a link between these two systems thus allowing the coordinates of the target volumes and critical structures to be defined relative to the treatment machine for treatment planning and the actual radiation treatment.

Another significant advance in the conformal technique is the use of electron accelerators for radiation treatment as compared to the high photon energy x-ray machines. The advantage of the several megavolts electron beam is that it deposits the ionizing energy preferentially at some predetermined depth in the tissue, thus sparing the skin and increasing the dose in the tumor.

The primary achievement of conformal therapy is a better local control of the disease that translates into longer survival rate of the patients. This better control is achieved by raising the radiation dose received by a tumor up to 80 Grays (Gy) while reducing injury to the critical structure around the tumor. For example, a significant reduction in long term morbidity was achieved by sparing the rectum and urethra during prostate cancer treatment.

Drawbacks of the external beam conformal radiation therapy are that it is a time consuming and expensive modality of radiation treatment. In addition, there is some significant room for improvement of the procedure and apparatus. For example, in case of prostate cancer conformal radiation treatment by an external radiation beam unavoidably delivers significant radiation doses to the prostate capsule and the neurovascular bundles responsible for erectile function and creates a long-term problem with potency.

An object of the current invention is to improve the quality of the conformal therapy and reduce cost of the radiation treatment.

Another object is to provide a highly automated high dose rate x-ray brachytherapy system.

Another object is to provide a radiation therapy system wherein the ionizing radiation comprises low energy x-rays in the range of energies 10-50 keV. Low energy x-rays provide very high gradients of the delivered dose, which can be instrumental in sparing the critical structures.

Another object is to provide better protection for medical personnel that perform radiation treatment. Low energy x-ray systems of the type contemplated for use in accord with the present invention do not require expensive bunker type radiation treatment facilities such as is required with radiation sources such as radioactive seeds. Thus, it is easier to protect medical personnel from unnecessary and damaging radiation exposure when performing a procedure using the apparatus and method of the present invention.

Another particular object is to avoid extensive irradiation of the urethra, rectum and cavernosal neurovascular bundles, responsible for the erectile function, thus sparing critical structures around prostate gland and avoiding associated morbidity and impotence.

### BRIEF DESCRIPTION OF THE INVENTION

The present invention provides apparatus and method for providing three dimensional conformal radiation therapy that enables a therapist to deliver a desired radiation dose to a target volume while reducing exposure of the surrounding tissue and critical structures. In one aspect of the present invention there will be provided an x-ray probe having proximal and distal ends and an x-ray emitter disposed at the distal end. The probe is mounted for translational and rotational motion relative to a support platform and is configured for insertion into the prostate gland. The support platform also mounts an ultrasound probe configured for insertion and operation in a patient's rectum; in operation the ultrasound probe is utilized to locate the x-ray probe relative to the previously identified target volume. The x-ray and ultrasound probes are operatively connected to a computer including a memory storing a target volume previously identified as well as radiation dose parameters. In operation the ultrasound probe will image the prostate gland and surrounding tissue and the computer will compare the resulting ultrasonic image with the previously identified target volume. Appropriate software within the computer will adjust the translational position of the x-ray probe to a first desired irradiation position and the x-ray emitter will be activated to deliver a desired irradiation dose at a first location relative to the target volume/prostate gland. Preferably the x-ray emitter will have a preferred narrow beam emission, enabling precise regions of the target volume to be targeted. The emitter can be rotated to sweep out a desired treatment volume and repositioned translationally. Dwelling times at each translational and rotational position will be determined prior to operation to ensure appropriate radiation dosages are received by the target volume while minimizing exposure of surrounding and critical tissues to the radiation. If desired, the x-ray probe can be positioned in multiple locations relative to the target volume to achieve a therapeutic treatment.

In another aspect of the present invention a method of treating a tumor is provided. A tumor and surrounding tissue are imaged using one or more of CAT scans, MRI, PET, or ultrasound. A target volume for treatment and a treatment regimen are determined including one or more locations for positioning an elongate x-ray probe having an x-ray emitter at its distal end relative to the target volume. An ultrasound probe is provided to locate the x-ray emitter relative to the target volume. The x-ray probe is movable translationally and rotationally to provide a predetermined therapeutic radiation dose to the target volume.

The foregoing objects and features of the present invention, as well as other various features and advantages, will become evident to those skilled in the art when the following description of the invention is read in conjunction with the accompanying drawings as briefly described below and the appended claims. Throughout the drawings, like numerals refer to similar or identical parts.

### BRIEF DESCRIPTION OF THE DRAWINGS

FIG. 1 illustrates an embodiment of the present invention.

FIG. 2 illustrates an enlarged view of the x-ray system and ultrasound imaging system shown in FIG. 1.

FIG. 3 illustrates in cross-section a target volume, specifically a prostate gland and surrounding tissue.

FIGS. 4A and 4B illustrates the co-ordinate systems used in the present invention to correlate prior imaging identifying a target treatment volume and the imaging system in use during a treatment procedure.

### DETAILED DESCRIPTION OF THE INVENTION

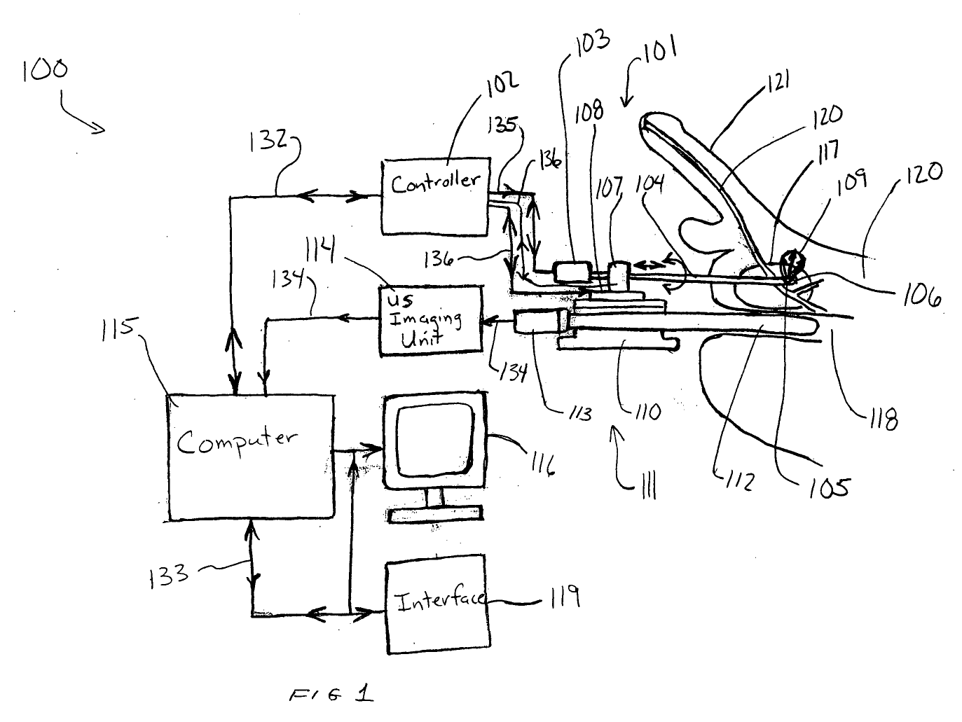

An embodiment of the invention comprising a system or apparatus for conformal radiation brachytherapy 100 is shown in FIG. 1. System 100 will be shown and described relative to therapeutic x-ray treatment of the prostate gland of a human male, though its use relative to other tumors will be understood.

The apparatus 100 comprises a therapeutic x-ray unit 101 including a controller 102, vacuum housing 103, elongated hollow probe 104 connected to the vacuum housing 103 and having an x-ray emitter 105 at its distal tip 106 generating a directional x-ray side beam 109. One type of x-ray generator, among others, useful in embodiment 100 is disclosed in U.S. patent application Ser. Nos. 10/392,1978 and 10/938,971, assigned to the same assignee as the present invention. The elongated probe 104 of the x-ray unit is secured to a rotational stage 107 that is, in its turn, connected to a linear stage 108 that during operation provides translational or longitudinal motion of the elongated probe (and the x-ray emitter 105 at its tip) along the probe axis. The rotational stage 107 during operation of the system provides rotational motion of the x-ray emitter and its side beam 109 around the axis of the elongated probe 104.

Rotational stage 107 communicates with the x-ray controller 102 via an appropriate connector 137 providing the controller with angular coordinates of the emitter beam 109 and receiving commands for further execution of the rotational motion. The linear stage 108 rests on a steady base 110 that is fastened to an operation table (not shown in FIG. 1). Linear stage 108 communicates with the x-ray controller 102 via an appropriate connector 136. Linear stage 108 provides translational or longitudinal coordinates of the x-ray beam and receives commands from the controller 102 about succeeding motions and dwelling times.

An ultrasound imaging system 111 comprises an imaging probe 112, electromechanical block 113 providing longitudinal and angular positioning of the ultrasound probe 112, ultrasound imaging unit 114 supplying a computer 115 with ultrasound imaging data and a display 116 providing image 2D slices and 3D imaging of the treated area of prostate 117 in the patient body 120. The ultrasound probe 112 is positioned in the patient's rectum 118. The penis 121 and urethra 122 of the patient are appropriately numbered. It will be understood that x-ray probe 104 as shown will be appropriately configured and structured for placement into the prostate gland 117 via the patient's perineum, though a urethral approach can also be utilized.

X-ray controller 102 communicates with the system computer 115 via an appropriate connector 132 while the ultrasound imaging unit 114 communicates with the same computer via an appropriate connector 134. Computer interface 119 is connected to the system computer 115 via an appropriate connector 133. The whole system is controlled by an operator from a computer interface 119.

In operation, a target volume will be identified in a patient by imaging with known or future medical imaging technologies. The coordinates of the target volume will be identified relative to the patient's body as well as the coordinate system of the imaging apparatus. Information regarding the target volume, including its coordinates and treatment protocols (dose rate, total dose, position, etc.) will be provided to the computer 115. The operational imager, such as the ultrasound probe 112, will be operationally placed into the proper position for imaging the target volume during a procedure and the probe 104 will be operationally placed relative to the target volume. The operational imager will image the probe 104 provide its coordinates relative to the imager to the computer 115, while will locate the probe relative to the target volume. The probe location can be adjusted translationally and rotationally and operated so as to provide the desired x-ray radiation therapy at the desired dose levels to the target volume.

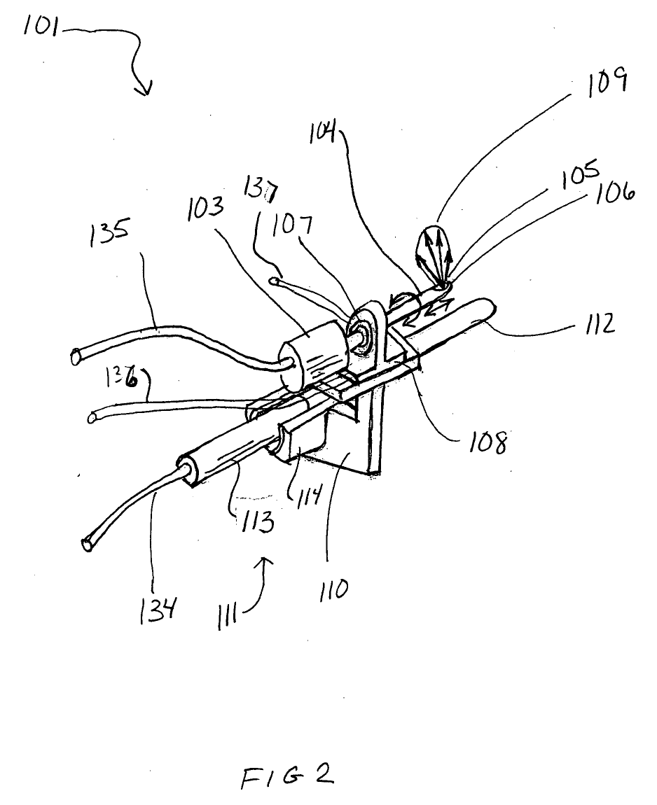

FIG. 2 shows an enlarged view of the x-ray system 101 and ultrasound imaging system 111 as they are secured to a base 110 during radiation treatment. The x-ray unit comprises housing 103, elongated probe 104 with an x-ray emitter 105 at its distal end that generates a side x-ray beam 109. The elongated probe 104 is secured to a rotation stage 107 providing rotation motion which ultimately is transferred to the x-ray beam 109 rotating around the longitudinal axis of the elongated probe 104. A linear translation stage 108 attached to immobile base 110 secured to the operation table (not shown). Stage 108 provides linear motion of the x-ray unit with the emitter 105 and side beam 109 along the longitudinal axis of the probe 104. The translation stage 108 communicates with the x-ray controller, not shown in this figure, via a cable 136. Translation stage 108 provides the x-ray controller with the current linear coordinates of the x-ray emitter and receives commands from the controller where to move and how long the dwelling time of the next position should be. In a similar manner the rotational stage 107 communicates with x-ray controller via a cable 137.

The ultrasound electromechanical block 113 is attached to a holder 114 that provides for linear and angular adjustment of the probe 112 position relative to the patient. The holder 114 is secured to the stationary base 110 attached to the operating table. Via a cable 134 the electromechanical block 113 communicates information to the system computer about current coordinates of the ultrasound beam and the intensity of the reflected from the tissues signal that allows reconstructing an ultrasound image of the treatment site in the system computer (not shown in the figure).

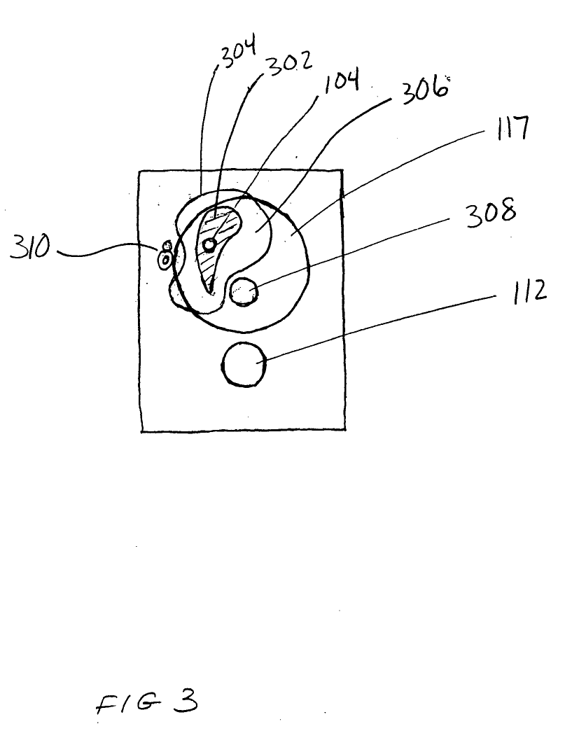

Treatment of a prostate gland tumor is shown in FIG. 3. FIG. 3 illustrates a cross sectional (slice) image of prostate gland 117 under treatment for a gross tumor 302. Tumor 302 is also shown in cross section and is encompassed by a contour line 304, which is the cross section of a 3D surface contouring the treatment volume 306. The critical structures of the prostate urethra 308 and cavernosal neurovascular bundle 310 are outside of the treatment volume and are supposed to get substantially lower dose than the treatment volume. The relative locations of the x-ray probe 104 and the ultrasound probe 112 within the patient's rectum 118 (shown in FIG. 1) are also shown.

It will be understood that the system 100 disclosed and discussed herein can be utilized to position the x-ray probe in a plurality of locations relative to the tumor 302, thus providing the therapist with the ability to irradiate the tumor and the target volume from multiple locations, multiple directions, and at multiple x-ray strengths so as to precisely tailor the therapy to provide the maximum dose to the target volume and reduced dosages to the tissues lying outside the target volume. The pattern of the plurality of locations can be selected in the diagnostic stage to maximize the therapeutic effects of the therapy and need not follow any preconceived template of or geometric pattern.

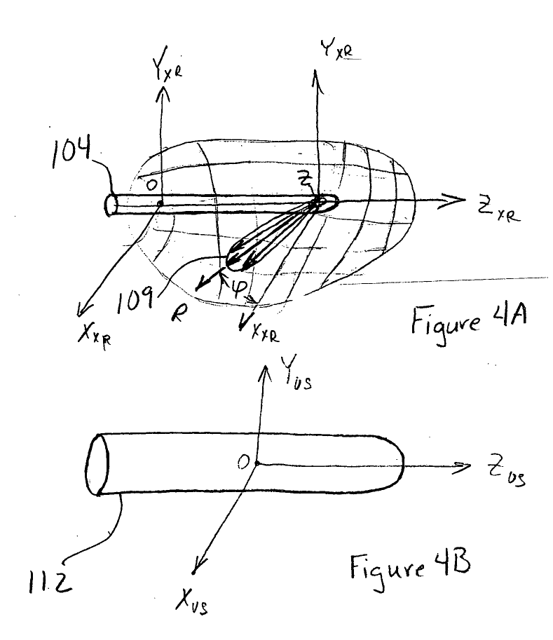

FIGS. 4A and 4B shows two coordinate systems used in the current invention for imaging and radiation treatment. The coordinate system 4A with orthogonal axes X_{XR}, Y_{XR}, and Z_{XR} is associated with the x-ray probe 104 and the coordinate system 4B with orthogonal axes X_{US}, Y_{US}, and Z_{US} is associated with the ultrasound imaging system. Both coordinate systems are immobile relative to the base 110 on which they are mounted and ultimately are related to the operation table. The difference between them is that they are shifted in spatial and angular positions relative to each other. The function of the ultrasound imaging system is to create an image of the treatment site including fiducial marks of the patient anatomy and/or special marks made on the skin of the patient. The ultrasound image includes also an image of the x-ray elongated probe positioned in the treatment site. The main computer of the system has in its memory a previously imported image of the treatment site with the 3D surface contouring the target volume 400 identified as the tissue within a 3d surface. This image and the 3D surface were created during diagnostic phase of the treatment and the development of the treatment plan. The image may be compiled from several images representing different imaging modalities like MRI, PET etc. Knowledge of 3D coordinates of fiducial marks of the patient's body and the coordinates of the x-ray probe allows transferring the therapeutic image from the computer memory to the coordinate system of the x-ray probe 104. The angular position φ of the x-ray beam is predetermined before the start of the radiation treatment and the starting z-position of the beam is known from the information provided by the current ultrasound image.

Having the image of the treatment volume correctly placed into the x-ray probe coordinate system, an initial angular coordinate φ and linear coordinate z of the beam, the main computer 115 of the system 100 after a command from the operator can execute an algorithm for irradiating the 3D surface of the target volume 400 with a predetermined level of radiation dose. The algorithm includes using known parameters of the beam: direction in 3D space, dose rate, and a radial function describing decreasing the dose rate with radial distance due to absorption in tissue (depth of penetration), which in its turn is defined by the operating voltage of the x-ray emitter. The algorithm selects dwelling times for the x-ray beam with a given angular and linear coordinates to deliver to the 3D surface contouring the treatment volume a predetermined dose.

The present invention has been described in language more or less specific as to the apparatus and method features. It is to be understood, however, that the present invention is not limited to the specific features described, since the apparatus and method herein disclosed comprise exemplary forms of putting the present invention into effect. For example, while an ultrasound probe has been illustrated as being the operational, real-time imaging apparatus during a therapeutic procedure, other compact imaging devices may appear in the near future and such would also be usable in accord with the present invention provided such use would be within acceptable safety considerations for a therapeutic procedure. The invention is, therefore, claimed in any of its forms or modifications within the proper scope of the appended claims appropriately interpreted in accordance with the doctrine of equivalency and other applicable judicial doctrines.

What is claimed is:

1. A system providing conformal x-ray brachytherapy for treatment of tumors by irradiation of a target volume of tissue in a patient, said system comprising:
an x-ray probe including an x-ray emitter;
an imaging probe configured to image the target volume;
a support base mounting said x-ray and imaging probes in known relation to each other;
a translation stage mounting said x-ray probe for translational motion;
a rotation stage mounting said x-ray probe for rotational motion; and
a computer operatively connected to said x-ray and imaging probes and said rotation and translation stages, said computer directing translational and rotational motion of said x-ray probe.

2. The system of claim 1 wherein the tumor is located in a prostate gland.

3. The system of claim 1 wherein said x-ray probe is elongated and configured for insertion into a prostate gland of a patient.

4. The system of claim 1 wherein said x-ray probe is configured for insertion into a prostate gland of a patient through the perineum.

5. The system of claim wherein said x-ray probe emits a side directional x-ray beam.

6. The system of claim 1 wherein said imaging probe is an ultrasound probe.

7. The system of claim 1 wherein said x-ray probe and said imaging probe each define coordinate systems.

8. The system of claim 1 and further including means for adjusting the translational and rotational position of said imaging probe.

9. The system of claim 1 and further including said system disposing said x-ray probe at multiple locations within the target volume.

10. A method providing conformal x-ray brachytherapy for treatment of tumors by irradiation of a target volume of tissue in a patient, said method comprising:
providing a three-dimensional image of the tumor;
providing an x-ray probe including an x-ray emitter translationally and rotationally movable;
providing an imaging probe for providing real-time imaging of the x-ray probe during a therapeutic procedure;
providing a computer for controlling the translational and rotational position of the x-ray probe during a procedure and the radiation dose emitted by the x-ray probe;
disposing the x-ray probe in proximity of the target volume; and
irradiating the target volume according to predetermined therapeutic protocols.

11. The method of claim 10 wherein the three-dimensional image of the tumor is provided by at least one of a CAT scan, PET scan, MRI, or ultrasound imaging.

12. The method of claim 10 wherein the x-ray probe and the imaging probe each define a coordinate system and wherein said method includes establishing a relationship between the coordinate systems for controlling the position of the x-ray probe.

13. The method of claim 10 wherein the tumor is in a prostate gland.

14. The method of claim 10 and further including translating and rotating the x-ray probe to multiple spatial locations to provide irradiation of the target volume.

15. The method of claim 10 and further including making multiple insertions of the x-ray probe into the tumor.
